(19) United States
(12) Patent Application Publication
(10) Pub. No.: US 2026/0226513 A1
(43) Pub. Date: Aug. 6, 2026

(54) NOVEL ACETYL-TRANSFERASES

(71) Applicant: DSM IP Assets B.V., Maastricht (NL)

(72) Inventors: Valmik Kanubhai VYAS, Winchester, MA (US); René Marcel DE JONG, Amsterdam (NL); Anna SYMBOR-NAGRABSKA, Reading, MA (US); Peter Louis HOUSTON, Boston, MA (US)

(21) Appl. No.: 19/150,263

(22) PCT Filed: Jan. 29, 2024
(86) PCT No.: PCT/EP2024/052033

§ 371 Date: Jul. 23, 2025

Related U.S. Application Data
(60) Provisional application No. 63/482,148, filed on Jan. 30, 2023.

(30) Foreign Application Priority Data
May 25, 2023 (EP) 23175417.7

(51) Int. Cl. C12P 7/62; C12N 1/18; C12N 9/02; C12N 9/10; C12R 1/85

(52) U.S. Cl. CPC C12P 7/62; C12N 1/18; C12N 9/0069; C12N 9/1029; C12R 2001/85; C12Y 113/11063; C12Y 203/01084

(57) ABSTRACT

The present invention is related to production of retinyl acetate generated via enzymatic conversion of retinol, said process including the use of modified enzymes with improved activity.

The present invention is related to production of retinyl acetate generated via enzymatic conversion of retinol, said process including the use of modified enzymes with improved activity.

Retinyl acetate is an important intermediate or precursor for production of retinoids, particularly such as vitamin A. Retinoids, including vitamin A, are one of very important and indispensable nutrient factors for human and animals which must be supplied via diet. Retinoids promote well-being, inter alia in respect of vision, the immune system and growth.

Current chemical production methods for retinoids, particularly vitamin A and precursors thereof, have some undesirable characteristics such as e.g. high-energy consumption, complicated purification steps and/or undesirable by-products. Therefore, over the past decades, other approaches to manufacture retinoids, particularly vitamin A and precursors thereof, have been investigated, including microbial conversion steps, which would be more economical as well as ecological.

In general, the biological systems that produce retinoids are industrially intractable and/or produce the compounds at such low levels that its isolation on industrial scale is not practicable of economic interest. There are several reasons for this, including instability of the retinoids in such biological systems or the relatively high production of by-products.

Acetylation of carotenoids, such as e.g. astaxanthin or zeaxanthin, by action of Atf1 from Saccharomyces bayanus has been previously reported (WO2014096992), with acetylation of for instance zeaxanthin in the range of up to 90%. However, these acetyl transferase enzymes usually have different substrate specificities for different alcohol substrates, which is determined by the local structural environment of the alcohol function on the molecule that is to be acetylated. For example, the hydroxy group to be acetylated in carotenoids, such as e.g. zeaxanthin, is located on the beta-ionone ring structure, whereas the hydroxy group to be acetylated in retinol is not located on the ionone ring structure but located at the other end on the CH₂ carbon at the end of the polyene chain of the molecule. Due to this different local molecular context of the acetylated hydroxy groups it is very difficult to make predictions on acetylation of retinols from data on acetylation of carotenoids.

For acetylation of retinoids, it turned out that an enzyme, i.e. acetyl transferase, originated from Lachancea, particularly Lachancea mirantina, i.e. LmATF1, is especially useful for acetylation of retinol into retinyl acetate. Using the wild-type LmATF in a retinol-producing strain of Yarrowia lipolytica, up to 40 wt % retinyl acetate (based on total retinoids) could be achieved. By substituting certain amino acids, an increase to more than 80 wt % was reached (see WO2020141168).

However, in order to use such enzymatic process at an industrial level, the efficiency towards production of retinyl acetate has to be further improved.

Surprisingly, we now could identify amino acid positions in fungal acetyl transferase, particularly ATF-enzyme originated from Lachancea mirantina as disclosed in WO2019058001, which are critical for formation of acetylated retinoids, particularly conversion of retinol into retinyl acetate. Modification of certain amino acids results in increased formation of retinyl acetate as comparted to the respective wild-type enzymes, such as e.g. an increase of at least about 10-20% when compared to acetylation of retinol using the respective non-modified enzymes, e.g. wt-ATF1 from Lachancea mirantina as disclosed in WO2019058001. Particularly, the percentage of retinyl acetate could be even further improved as compared to the known best enzymes so far, such as disclosed in WO2020141168.

Particularly, the present invention is related to a modified enzyme and a method for production of said modified enzymes involved in acetylation of retinol into retinyl acetate in a suitable retinol-producing host cell with a percentage of at least about 81% retinyl acetate based on total retinoids, particularly fungal enzyme comprising one or more modification(s), such as amino acid substitution(s), in a sequence with at least about 20%, such as 25, 30, 35, 40, 45, 50, 55, 60, 65, 70, 75, 80, 85, 90, 92, 95, 97, 98, 99% or up to 100% identity to SEQ ID NO:1 (FIG. 1) or SEQ ID NO:3 (FIG. 2), said one or more amino acid substitution(s) being located at position(s) corresponding to amino acid residue(s) selected from the group consisting of position 68, 451, 452, 473, 483, 512 and combinations thereof, in the polypeptide according to SEQ ID NO:1, particularly comprising at least one amino acid substitution on position corresponding to A451, T473, and/or L483 in the polypeptide according to SEQ ID NO:1, wherein upon introduction of said amino acid substitution(s) the percentage of retinyl acetate is increased by at least 10-20 wt % as compared to the process using the same conditions but the respective or corresponding wild-type enzyme, including an ATF enzyme according to SEQ ID NO:1.

The use of such modified enzyme in a process for production of retinoids, wherein said modified enzyme is expressed, particularly heterologous expressed, in a suitable host cell, particularly fungal host cell capable of retinol production, leads to an increase in retinyl acetate in the range of at least about 10% based on total retinoids present in/produced by the modified host cell as compared to the process using the same conditions but an ATF enzyme according to SEQ ID NO:1.

The terms “acetyl transferase”, “retinol acetylating enzyme”, “enzyme having retinol acetylating activity”, “ATF” or “ATF1” are used interchangeably herein and refer to enzymes of EC class [EC 2.3.1.84] which are capable of catalyzing the conversion of retinol into retinyl acetate, with particularly about 30 to 90 wt % in the acetylated form based on total retinoids, including both naturally occurring enzymes and enzymes synthetically generated by the help of artificial intelligence. Such enzyme as used herein is called a “non-modified” ATF. Examples of such non-modified enzymes are shown in SEQ ID NO: 1 or 3, e.g. enzymes isolated or derivable from Lachancea mirantina (LmATF1) as shown in FIG. 1 or 2.

A “modified” ATF as defined herein and particularly based on “non-modified” ATF, such as e.g. an enzyme with at least about 20% identity to SEQ ID NO:1 or SEQ ID NO:3, shows an increase in the formation of retinyl acetate from conversion of retinol, such as particularly an increase of at least about 10% based on total retinoids and as compared to retinyl acetate formation using an enzyme according to SEQ ID NO:1.

Suitable non-modified enzymes including enzymes with at least about 20%, such as 25, 30, 35, 40, 45, 50, 55, 60, 65, 70, 75, 80, 85, 90, 92, 95, 97, 98, 99% or up to 100% identity to SEQ ID NO:1 or 3, including LmATF isolated/originated from Lachancea mirantina, are obtainable from fungal enzymes comprising a highly conserved partial amino acid sequence of at least 7 amino acid residues selected from N-H-x(3)-D-[GA](motifs are in Prosite syntax, as defined in https://lprosite.expasy.org/scanprosite/scanprosite_doc.html), wherein “x” denotes an arbitrary amino acid and with the central histidine being part of the enzyme's binding pocket, preferably wherein the 7 amino acid motif is selected from NHCSSDG, NHCLCDG or NHILKDG, more preferably selected from NHCSSDG corresponding to position N218 to G224 in the polypeptide according to SEQ ID NO:1.

Modified ATFs as defined herein are capable of converting retinol into retinyl acetate, particularly with conversion ratios being increased by at least about 10% as compared to conversion of retinol into retinyl acetate using the non-modified enzyme according to SEQ ID NO:1, such as e.g. obtainable via expression of a modified ATF under suitable culture conditions including but not limited to cultivation on glucose, galactose, xylose with or without the combination of ethanol.

The enzymes as defined herein are used in the conversion of retinol into retinyl acetate, wherein the substrate (i.e. retinol) can be either cis-, trans- or a mix of cis-/trans-retinol in any possible ratio. Preferably, the retinol-mix to be used as substrate has high percentage of trans-retinol, such as e.g. at least about 65 to 98 wt % of trans-isomer based on total retinol in the host cell. Acetylation of said retinol-mix with at least about 65 to 98 wt % trans-retinol would lead to retinyl acetate with about the same ratio of trans to cis-retinyl acetate based on total retinyl acetate produced by the host cell.

In one specific embodiment, the present invention is related to conversion of retinol into retinyl acetate using a suitable host cell as defined herein comprising and expressing a modified enzyme as defined herein, wherein the retinol is a mix of trans- and cis-retinol and wherein the percentage of trans-retinol is in the range of at least about 65 to 98 wt % trans retinol based on total retinol.

The terms “conversion”, “enzymatic conversion”, “acetylation” or “enzymatic acetylation” in connection with enzymatic catalysis of retinol are used interchangeably herein and refer to the action of modified or non-modified ATF in the catalysis of retinol to retinyl acetate conversion resulting in a certain percentage of retinyl acetate based on total retinoids present/produced by a suitable host cell upon expression of said ATF, wherein an increase by at least 10% in retinyl acetate based on total retinoids can be achieved using a modified ATF as defined herein.

Suitable host cells according to the present invention include fungal host cells as well as cells from e.g. E. coli. As used herein, the term “fungal host cell” particularly includes yeast cells, wherein the cell is a retinol-producing host cell, particularly a retinyl acetate-producing host cell, such as retinyl acetate-producing fungal host cell, including but not limited to Yarrowia or Saccharomyces, such as e.g. Yarrowia lipolytica or Saccharomyces cerevisiae.

The modified ATF enzyme might be used in an isolated form (e.g. in a cell-free system) or might be expressed in the suitable host cell, such as e.g. retinol-producing host cell, particularly fungal host cell as defined herein. Enzymes might be expressed as endogenous enzymes or as heterologous enzymes. Preferably, the modified enzymes as described herein are introduced and expressed as heterologous enzymes in a suitable host cell, such as e.g. a retinol-producing host cell, particularly fungal host cell as defined herein.

In one embodiment, the modified ATF enzyme as defined herein to be used for production of retinyl acetate as defined herein comprises an amino acid substitution at a position corresponding to residue 68 in the polypeptide according to SEQ ID NO:1 or 3 leading to leucine at said residue, such as e.g. via substitution of glutamine by leucine (Q68L). Said modified enzyme might be derived from Lachancea, such as e.g. L. mirantina, L. fermentati, preferably from L. mirantina. Using such modified enzyme comprising said mutation in a fermentation process using a suitable carbon source such as e.g. glucose results in an increase of at least about 10%, such as e.g. 15, 20, 25, 30, 35, 40, 45, 50, 55, 60, 65, 70, 75, 80, 85, 90, 95, 100, 105, 110, 115, 120, 125, 130, 135, 140, 145% or more, retinyl acetate based on total retinoids from acetylation of retinol compared to the corresponding process using an enzyme according to SEQ ID NO:1. The mutation might furthermore be combined with additional mutation(s) as defined herein, such as particularly with one or more amino acid substitution(s) at position(s) corresponding to residue(s) 451 and/or 452 and/or 473 and/or 483 and/or 512 in the polypeptide according to SEQ ID NO:1 or 3.

In one embodiment, the modified ATF enzyme as defined herein to be used for production of retinyl acetate as defined herein comprises an amino acid substitution at a position corresponding to residue 451 in the polypeptide according to SEQ ID NO:1 or 3 leading to leucine or methionine at said residue, such as e.g. via substitution of alanine by leucine (A451L) or alanine by methionine (A451M). Said modified enzyme might be derived from Lachancea, such as e.g. L. mirantina, L. fermentati, preferably from L. mirantina. Using such modified enzyme comprising said mutation in a fermentation process using a suitable carbon source such as e.g. glucose results in an increase of at least about 20%, such as e.g. 25, 30, 35, 40, 45, 50, 55, 60, 65, 70, 75, 80, 85, 90, 95, 100, 120, 150, 200, 220, 250, 280, 300, 350, 400, 450, 500 or more, retinyl acetate based on total retinoids from acetylation of retinol compared to the corresponding process using an enzyme according to SEQ ID NO:1. The mutation might furthermore be combined with additional mutation(s) as defined herein, such as particularly with one or more amino acid substitution(s) at position(s) corresponding to residue(s) 68 and/or 452 and/or 473 and/or 483 and/or 512 in the polypeptide according to SEQ ID NO:1 or 3.

In one embodiment, the modified ATF enzyme as defined herein to be used for production of retinyl acetate as defined herein comprises an amino acid substitution at a position corresponding to residue 452 in the polypeptide according to SEQ ID NO:1 or 3 leading to phenylalanine at said residue, such as e.g. via substitution of leucine by phenylalanine (L452F). Said modified enzyme might be derived from Lachancea, such as e.g. L. mirantina, L. fermentati, preferably from L. mirantina. Using such modified enzyme comprising said mutation in a fermentation process using a suitable carbon source such as e.g. glucose results in an increase of at least about 20%, such as e.g. 25, 30, 35, 40, 45, 50, 55, 60, 65, 70, 75, 80, 85, 90, 95, 100, 120, 150, 200, 220, 250, 280, 300, 350, 400, 450, 500 or more, retinyl acetate based on total retinoids from acetylation of retinol compared to the corresponding process using an enzyme according to SEQ ID NO:1. The mutation might furthermore be combined with additional mutation(s) as defined herein, such as particularly with one or more amino acid substitution(s) at position(s) corresponding to residue(s) 68 and/or 451 and/or 473 and/or 483 and/or 512 in the polypeptide according to SEQ ID NO:1 or 3.

In one embodiment, the modified ATF enzyme as defined herein to be used for production of retinyl acetate as defined herein comprises an amino acid substitution at a position corresponding to residue 473 in the polypeptide according to SEQ ID NO:1 or 3 leading to leucine or alanine at said residue, such as e.g. via substitution of threonine by leucine (T473L) or threonine by alanine (T473A). Said modified enzyme might be derived from Lachancea, such as e.g. L. mirantina, L. fermentati, preferably from L. mirantina. Using such modified enzyme comprising said mutation in a fermentation process using a suitable carbon source such as e.g. glucose results in an increase of at least about 20%, such as e.g. 25, 30, 35, 40, 45, 50, 55, 60, 65, 70, 75, 80, 85, 90, 95, 100, 120, 150, 200, 220, 250, 280, 300, 350, 400, 450, 500 or more, retinyl acetate based on total retinoids from acetylation of retinol compared to the corresponding process using an enzyme according to SEQ ID NO:1. The mutation might furthermore be combined with additional mutation(s) as defined herein, such as particularly with one or more amino acid substitution(s) at position(s) corresponding to residue(s) 68 and/or 451 and/or 452 and/or 483 and/or 512 in the polypeptide according to SEQ ID NO:1 or 3.

In one embodiment, the modified ATF enzyme as defined herein to be used for production of retinyl acetate as defined herein comprises an amino acid substitution at a position corresponding to residue 512 in the polypeptide according to SEQ ID NO:1 or 3 leading to phenylalanine at said residue, such as e.g. via substitution of asparagine by phenylalanine (N512F). Said modified enzyme might be derived from Lachancea, such as e.g. L. mirantina, L. fermentati, preferably from L. mirantina. Using such modified enzyme comprising said mutation in a fermentation process using a suitable carbon source such as e.g. glucose results in an increase of at least about 20%, such as e.g. 25, 30, 35, 40, 45, 50, 55, 60, 65, 70, 75, 80, 85, 90, 95, 100, 105, 110, 115, 120, 125, 130, 135, 140, 145, 150 or more, retinyl acetate based on total retinoids from acetylation of retinol compared to the corresponding process using an enzyme according to SEQ ID NO:1. The mutation might furthermore be combined with additional mutation(s) as defined herein, such as particularly with one or more amino acid substitution(s) at position(s) corresponding to residue(s) 68 and/or 451 and/or 452 and/or 473 and/or 483 in the polypeptide according to SEQ ID NO:1 or 3.

The host cell as described herein is capable of conversion of retinol into retinyl acetate with conversion ratios which are increased by at least about 10-20% or more compared to conversion via the respective enzyme according to SEQ ID NO:1, particularly with an increase in the range of 10-50% and more (based on the total amount of retinoids produced by said host cell) towards generation of retinyl acetate, such as e.g. obtainable via expression of a modified ATF under suitable culture conditions including but not limited to cultivation on glucose, galactose, xylose with or without the presence of ethanol, and a suitable host cell, such as e.g. selected from a fungal host cell including Yarrowia or Saccharomyces or other microbial host cell such as e.g. Escherichia coli. Suitable conditions might be cultivation in a fed-batch fermentation of e.g. 80, 90, 100, 110, 120, 130 h.

A modified host cell as defined herein comprises one or more copies of modified ATFs as defined herein, preferably wherein the ATFs are heterologous expressed in said modified host cell. Modifications in order to have the host cell as defined herein produce more copies of genes and/or proteins, such as e.g. more copies of modified ATFs with selectivity towards formation of retinyl acetate as defined herein, including increased conversion by at least about 10-20% as compared to a process using LmATF1 according to SEQ ID NO:1 (FIG. 1) based on the total amount of retinoids produced by said host cell towards generation of retinyl acetate, such as e.g. obtainable via expression of a modified ATF under suitable culture conditions including but not limited to cultivation on glucose, galactose, xylose with or without the presence of ethanol, may include the use of strong promoters, suitable transcriptional- and/or translational enhancers, or the introduction of one or more gene copies into the retinol-producing host cell, particularly fungal host cell, leading to increased accumulation of the respective enzymes in a given time. The skilled person knows which techniques to use depending on the host cell. The increase or reduction of gene expression can be measured by various methods, such as e.g. Northern, Southern or Western blot technology as known in the art.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

Thus, the present invention is directed to a retinol-producing host cell, particularly fungal host cell, as described herein comprising an expression vector or a polynucleotide encoding modified ATFs, as described herein which has been integrated in the chromosomal DNA of the host cell. Such retinol-producing host cell, particularly fungal host cell, comprising a heterologous polynucleotide either on an expression vector or integrated into the chromosomal DNA encoding modified ATFs as described herein is called a recombinant or modified host cell. The retinol-producing host cell, particularly fungal host cell, might contain one or more copies of a gene encoding the modified ATFs as defined herein, comprising the mutations as defined herein, leading to overexpression of such genes encoding said modified ATFs, particularly Atf1 enzymes, as defined herein. The increase of gene expression can be measured by various methods, such as e.g. Northern, Southern or Western blot technology as known in the art.

The present invention is particularly directed to the use of such novel modified ATF enzymes, in a process for production of retinyl acetate, particularly under conditions wherein the amount of other retinyl esters, particularly long-chain retinyl esters is reduced. The skilled person knows how to generate such conditions (see, e.g. WO2021136689 or WO2022090548). Retinyl acetate can be further converted into vitamin A by the action of (known) suitable chemical or biotechnological mechanisms.

The terms “sequence identity”, “% identity” are used interchangeable herein. For the purpose of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity.

The modified ATF enzymes as defined herein also encompass enzymes carrying further amino acid substitution(s) which do not alter enzyme activity, i.e. which show the same properties with respect to the enzymes defined herein and catalyze the conversion of retinol to retinyl acetate sa described herein. Such mutations are also called “silent mutations”, which do not alter the (enzymatic) activity of the enzymes according to the present invention.

Expression of the enzymes/polynucleotides encoding one of the modified enzymes, as defined herein can be achieved in any host system, including (micro)organisms, which is suitable for retinoid (including retinol) production and which allows expression of the nucleic acids encoding one of the enzymes as disclosed herein, including functional equivalents or derivatives as described herein. Examples of suitable retinol-producing host (micro)organisms are bacteria, algae, fungi, including yeasts, plant or animal cells. Preferred bacteria are those of the genera Escherichia, such as, for example, Escherichia coli, Streptomyces, Pantoea (Erwinia), Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis, Paracoccus, such as, for example, Paracoccus zeaxanthinifaciens. Preferred eukaryotic microorganisms, in particular fungi including yeast, are selected from Saccharomyces, such as Saccharomyces cerevisiae, Aspergillus, such as Aspergillus niger, Pichia, such as Pichia pastoris, Hansenula, such as Hansenula polymorpha, Kluyveromyces, such as Kluyveromyces lactis, Phycomyces, such as Phycomyces blakesleanus, Mucor, Rhodotorula, Sporobolomyces, Xanthophyllomyces, Phaffia, Blakeslea, such as e.g. Blakeslea trispora, or Yarrowia, such as Yarrowia lipolytica. In particularly preferred is expression in a fungal host cell, such as e.g. Yarrowia or Saccharomyces, or expression in Escherichia, more preferably expression in Yarrowia lipolytica or Saccharomyces cerevisiae.

Depending on the host cell the polynucleotides as defined herein for acetylation of retinol might be optimized for expression in the respective host cell. The skilled person knows how to generate such further modified polynucleotides. It is understood that the polynucleotides as defined herein also encompass such host-optimized nucleic acid molecules as long as they still express the polypeptide with the respective activities as defined herein.

Thus, in one embodiment, the present invention is directed to a retinol-producing host cell, particularly fungal host cell, comprising polynucleotides encoding modified ATF enzymes as defined herein which are optimized for expression in said host cell and which are used for production of retinyl acetate. Particularly, a retinol-producing host cell, particularly fungal host cell, is selected from yeast, e.g. Yarrowia or Saccharomyces, such as e.g. Saccharomyces cerevisiae or Yarrowia lipolytica, wherein the polynucleotides encoding the modified ATF enzymes as defined herein are selected from polynucleotides expressing modified polypeptides comprising one or more amino acid substitution(s) in a sequence with at least 20%, such as e.g. 25, 30, 35, 40, 45, 50, 55, 60, 65, 70, 75, 80, 85, 90, 92, 95, 97, 98, 99% or up to 100% identity to SEQ ID NO:1 or 3, such as e.g. introduction of one or more amino acid substitution(s) at position(s) corresponding to residue(s) selected from the group consisting of position 68, 451, 452, 473, 483, 512, and combinations thereof and as defined herein, particularly comprising at least one amino acid substitution on position corresponding to A451, T473, and/or L483 in the polypeptide according to SEQ ID NO:1 (FIG. 1), wherein upon introduction of said amino acid substitution(s) the percentage of retinyl acetate is increased by at least 10-20 wt % as compared to the process using the same conditions but the respective or corresponding wild-type enzyme, including an ATF enzyme according to SEQ ID NO:1, and preferably comprising a highly conserved partial amino acid sequence, i.e. common active site or “Prosite-motif”, of at least 7 amino acid residues selected from N-H-x(3)-D-G corresponding to position N218 to G224 in the polypeptide according to SEQ ID NO:1 (motifs are in Prosite syntax, as defined in https://prosite.expasy.org/scanprosite/scanprosite_doc.html) and wherein “x” denotes an arbitrary amino acid, said host cell producing retinyl acetate with an increase of at least about 10% and compared to a host cell expressing an enzyme according to SEQ ID NO:1, such as e.g. obtainable via expression of a modified ATF under suitable culture conditions including but not limited to cultivation on glucose, galactose or xylose.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code). Thus, for example, strain Lachancea mirantina is a synonym of strain Zygosaccharomyces sp. IFO 11066, originated from Japan.

The present invention is directed to a process for production of retinyl acetate, wherein the retinyl acetate is generated via acetylation of retinol (particularly at least 65% as trans-retinol) as disclosed herein by the action of modified ATF enzymes as described herein, wherein the acetylating enzymes are preferably heterologous expressed in a suitable host cell under suitable conditions as described herein. The produced retinyl acetate might be isolated and optionally further purified from the medium and/or host cell. Said acetylated retinoids defined herein can be used as building blocks in a multi-step process leading to vitamin A. Vitamin A might be isolated and optionally further purified from the medium and/or host cell as known in the art.

Preferably, acetylation of retinol by the use of modified ATFs as described herein, can lead ot increased titers of retinyl acetate, such as e.g. at least about 50 to 92 wt % retinyl acetate based on total retinods, i.e. percentage in the range of at least about 50 to 92%, such as 55, 60, 65, 70, 75, 80, 85, 90% or more of acetylated retinoids, i.e. retinyl acetate, based on total retinoids present in the retinoid mix produced by the host cell, such as e.g. obtainable via expression of a modified enzymes under suitable culture conditions including but not limited to cultivation on glucose, galactose or xylose. In a more preferred embodiment, a retinol mix with a percentage of at least about 65% trans-retinol is used as substrate for acetylation via the modified enzymes as defined herein.

The host cell, i.e. microorganism, algae, fungal, animal or plant cell, capable of producing retinol, might furthermore be capable of production of beta-carotene, which might be furthermore enzymatically converted into retinal which might be furthermore converted into retinol. The skilled person knows which genes to be used/expressed for either biosynthesis of beta-carotene and/or bio-conversion of beta-carotene into retinol. Such host cell further being capable of expressing the modified ATFs as defined herein, and/or further genes required for biosynthesis of vitamin A, may be cultured in an aqueous medium supplemented with appropriate nutrients under aerobic or anaerobic conditions and as known by the skilled person for the respective retinol-producing host cells. Optionally, such cultivation is in the presence of proteins and/or co-factors involved in transfer of electrons, as known in the art. Suitable carbon sources for the purpose of the present invention might be selected from glucose, fructose, raffinose, lactose, galactose, glycerol, xylose, arabinose, sucrose or maltose with or without the presence of ethanol, particularly selected from glucose, galactose or xylose. Particular culture conditions might contain a batch and feed run, with a concentration of 5% (w/v) glucose and 1% ethanol (w/v) in the batch phase and a concentration of 100% (w/v) in the feeding phase. The cultivation/growth of the host cell may be conducted in batch, fed-batch, semi-continuous or continuous mode, particularly in fed-batch mode for 80, 90, 100, 110, 120, 130 h under suitable culture conditions. Depending on the host cell, preferably, production of retinoids such as e.g. vitamin A, precursors and/or derivatives thereof such as retinal, retinol, retinyl esters, particularly retinyl acetate, can vary, as it is known to the skilled person. Cultivation and isolation of beta-carotene and retinoid-producing host cells selected from Yarrowia and Saccharomyces is described in e.g. WO2008042338. With regards to production of beta-carotene and retinoids in host cells selected from E. coli, methods are described in e.g. US20070166782.

Particularly, the fermentation using suitable retinoid-producing host strains as defined herein expressing the modified ATFs as described herein are cultivated in a two-phase system, wherein the retinoids, including but not limited to retinyl acetate, are collected in and afterwards isolated from a suitable lipophilic phase. Particular conditions and lipophilic solvents are disclosed in WO2022090548 or WO2022090549.

In some embodiments, the present invention is directed to a two-phase fermentation using a lipophilic solvent as second phase, such as e.g. isopars or corn oil, besides the known solvents comprising Drakeol®, silicone or n-dodecane (see Jang et al., Microbial Cell Factories 10:59, 2011).

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a suitable (cell-free) system in the presence of a suitable substrate. The skilled person knows how to measure enzyme activity, Analytical methods to evaluate the capability of a suitable ATF, particularly Atf1, as defined herein for retinyl acetate production, from conversion of retinol are known in the art, such as e.g. described in Example 4 of WO2014096992. In brief, titers of products such as retinyl acetate, retinol, trans-retinal, cis-retinal, beta-carotene and the like can be measured by HPLC.

With regards to suitable host cells comprising specific enzymes involved in biosynthesis of beta-carotene and that are expressed and active in vivo leading to production of carotenoids, e.g. beta-carotene, both genes and methods to generate carotenoid-producing host cells are known in the art, see e.g. WO2006102342. Depending on the carotenoid to be produced, different genes might be involved.

As used herein, a “retinol-producing host cell” is a host cell, wherein the respective polypeptides are expressed and active in vivo, leading to production of retinoids, e.g. vitamin A and its precursors including retinol, via enzymatic conversion of beta-carotene via retinal into retinol. These polypeptides include the modified ATFs as defined herein. The genes of the vitamin A pathway and methods to generate retinoid-producing host cells are known in the art. The term retinoid includes retinol, which is used as a substrate for the modified acetylating enzymes as defined herein.

Retinoids as used herein include beta-carotene cleavage products also known as apocarotenoids, including but not limited to retinal, retinolic acid, retinol, retinoic methoxide, retinyl acetate, retinyl esters, 4-keto-retinoids, 3 hydroxy-retinoids or combinations thereof. Long chain retinyl esters as used herein are defined as hydrocarbon esters of retinol with fatty acids, where the fatty acids consist of at least about 8, such as e.g. 9, 10, 12, 13, 15 or 20 carbon atoms and up to about 26, such as e.g. 25, 22, 21 or less carbon atoms, with preferably up to about 6 unsaturated bonds, such as e.g. 0, 1, 2, 4, 5, 6 unsaturated bonds. The fatty acids in the long chain retinyl esters include but are not limited to linoleic acid, oleic acid or palmitic acid. Biosynthesis of retinoids is described in e.g. WO2008042338.

“Retinal” as used herein is known under IUPAC name (2E,4E,6E,8E)-3,7-Dimethyl-9-(2,6,6-trimethylcyclohexen-1-yl)nona-2,4,6,8-tetraenal. It is herein interchangeably referred to as retinaldehyde or vitamin A aldehyde and includes both cis- and trans-isoforms, such as e.g. 11-cis retinal, 13-cis retinal, trans-retinal and all-trans retinal.

The term “carotenoids” as used herein is well known in the art. It includes long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006102342.

“Vitamin A” as used herein may be any chemical form of vitamin A found in aqueous solutions, in solids and formulations, and includes retinol, retinyl acetate and retinyl esters. It also includes retinoic acid, such as for instance undissociated, in its free acid form or dissociated as an anion.

Particularly, the present invention is directed to the following embodiments (1) to (14):

(1) Modified acetyl transferase [EC 2.3.1.84] with increased catalytic activity towards acetylation of retinol, wherein the enzyme is based on an enzyme with at least 20% identity to Lachancea mirantina ATF1 according to SEQ ID NO:1 or SEQ ID NO:3, said modified enzyme comprising a 7 amino acid motif N-H-x(3)-D-[GA], wherein “x” denotes an arbitrary amino acid, and wherein said motif corresponding to position N218 to G224 in the polypeptide according to SEQ ID NO:1, said modified acetyl transferase comprising one or more amino acid substitution(s) at position(s) corresponding to Q68, A451, L452, T473, L483 and/or N512 in the non-modified polypeptide according to SEQ ID NO:1, said modified enzyme being used in acetylation of retinol into retinyl acetate with a percentage of at least about 81% retinyl acetate based on total retinoids.

(2) Modified enzyme according to embodiment (1), wherein the glutamine on position corresponding to 68 in SEQ ID NO:1 is replaced by leucine, and/or wherein the alanine on position corresponding to 451 in SEQ ID NO:1 is replaced by leucine or methionine, and/or wherein the leucine on position corresponding to 452 in SEQ ID NO:1 is replaced by phenylalanine, and/or wherein the threonine on position corresponding to 473 in SEQ ID NO:1 is replaced by leucine or alanine, and/or wherein the leucine on position corresponding to 483 in SEQ ID NO:1 is replaced by methionine, and/or wherein the asparagine on position corresponding to 512 in SEQ ID NO:1 is replaced by phenylalanine.

(3) Modified enzyme according to embodiment (1) or (2), furthermore comprising one or more amino acid substitution(s) at position(s) corresponding to amino acid residues selected from H69, V407, G409, S480 and/or I484 in the non-modified polypeptide according to SEQ ID NO:3.

(4) Modified enzyme according to embodiment (3), wherein the histidine on position corresponding to 69 in SEQ ID NO:1 is replaced by alanine, asparagine or serine, and/or wherein the valine on position corresponding to 407 in SEQ ID NO:1 is replaced by isoleucine, and/or wherein the glycine on position corresponding to 409 in SEQ ID NO:1 is replaced by alanine, and/or wherein the serine on position corresponding to 480 in SEQ ID NO:1 is replaced by glutamic acid, phenylalanine, leucine, methionine or glutamine, and/or wherein the isoleucine on position corresponding to 484 in SEQ ID NO:1 is replaced by leucine.

(5) Modified enzyme according to embodiment (1), (2), (3) or (4), wherein the percentage of retinyl acetate based on total retinoids obtained from catalytic acetylation of retinol is increased by at least 8% compared to catalytic acetylation reaction using the corresponding non-modified enzyme according to SEQ ID NO:1.

(6) Modified enzyme according to embodiment (1), (2), (3), (4) or (5), which is expressed in a retinol-producing host cell expressing genes involved in catalysis of retinal into retinol and/or beta-carotene into retinal.

(7) Retinoid-producing host cell expressing an enzyme according to embodiment (1), (2), (3), (4), (5) or (6).

(8) Host cell according to embodiment (7) which is a fungal host cell. (9) Host cell according to embodiment (8) which is selected from Yarrowia or Saccharomyces.

(10) Host cell according to embodiment (7), (8) or (9), further expressing enzymes involved in the mevalonate pathway and/or carotenoid pathway for generation of beta-carotene, retinal and retinol.

(11) Host cell according to embodiment (10), wherein the enzyme catalyzing the conversion of beta-carotene into retinal is a beta carotene oxygenase selectively producing trans-retinal with a percentage of at least 95% trans-retinal based on total retinoids comprising cis- and trans-retinal.

(12) Process for the production of retinoids comprising retinal, retinol and retinyl acetate, comprising cultivation of the host cell according to embodiment (7), (8), (9), (10) or (11) under suitable culture conditions with the carbon source being selected from glucose, fructose, raffinose, lactose, galactose, glycerol, xylose, arabinose, sucrose, maltose, ethanol or mixture thereof and in the presence of a lipophilic substance, wherein the percentage of retinyl acetate generated during said process is at least 81% based on total retinoids.

(13) Process according to embodiment (12), wherein the lipophilc substance is selected from synthetic or natural oils or isoparaffins.

(14) Process according to embodiment (12) or (13), wherein the percentage of retinyl acetate based on total retinoids is increased by at least 8 to 500% compared to a process using an ATF1 according to SEQ ID NO:1.

### FIGURES

FIG. 1: Amino acid sequence of Lachancea mirantina ATF1 (LmATF; SEQ ID NO:1) wherein the residues selected for the amino acid substitutions as described in the present application are marked in bold/underlined and each tenth amino acid residue is marked in bold.

FIG. 2: Amino acid sequence of Lachancea mirantina ATF1 (LmATF*; SEQ ID NO:3) wherein the residues selected for the amino acid substitutions as described in the present application are marked in bold/underlined and each tenth amino acid residue is marked in bold.

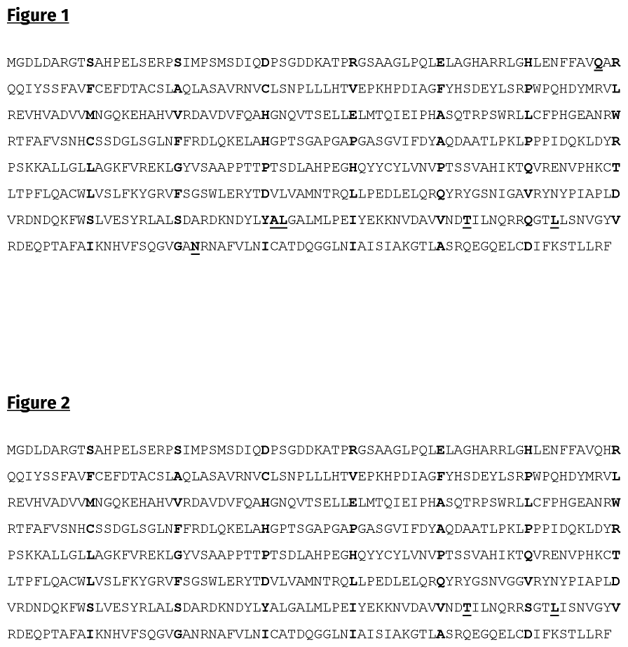

The following examples are illustrative only and are not intended to limit the scope of the invention in any way. The contents of all references, patent applications, patents, and published patent applications, cited throughout this application are hereby incorporated by reference, in particular WO2014096992, WO2019058001, WO2021136689, WO2022090548, WO2008042338, US20070166782, WO2022090549, WO2006102342, WO2020141168, and WO2016172282.

### EXAMPLES

### Example 1: General Methods, Strains, and Plasmids

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

Shake plate assay (Yarrowia). For testing the conversion activity of the mutants, typically, 200 μl of 0.25% Yeast extract, 0.5% peptone (0.25×YP) was inoculated with 10 μl of freshly grown Yarrowia and overlaid with 200 μl of mineral oil (Isopar M, Exxon Mobile) with 2% oleic acid as carbon source in the mineral oil phase. Transformants were grown in 24 well plates (Microplate Devices 24 Deep Well Plates Whatman 7701-5102), covered with mat seal (Analytical Sales and Services Inc. Plate Mats 24010CM), sterile sealed with Qiagen Airpore Tape Sheets (19571) and shaken in Infors multi plate shaker (Multitron), 30° C., 800 RPM for 4 days. The mineral oil fraction was removed from the shake plate wells and analyzed by UPLC reverse phase column, with a photo-diode array detector. This method is also used in Example 2.

DNA transformation. Yarrowia lipolytica strains were transformed from overnight growth on YPD plate media. 50 μl of cells was scraped from a plate and transformed by incubation in 500 μl with 1 μg transforming DNA, typically linear DNA for integrative transformation, 40% PEG 3550MW, 100 mM lithium acetate, 50 mM Dithiothreitol, 5 mM Tris-Cl pH 8.0, 0.5 mM EDTA for 30 minutes at 40° C. and plated directly to selective media or, in the case of dominant antibiotic marker selection, the cells were out grown on YPD liquid media for 4 hours at 30° C. before plating on the selective media. Saccharomyces strains were transformed using the lithium-acetate method from exponential phase YPD-grown cells, which were grown by subculture of an overnight YPD culture. 10⁸ cells/transformation were harvested and resuspended in a mixture containing 40% PEG 3350 (MW), 100 mM lithium acetate, 10 mM Tris-Cl pH 8.0, 1 mM EDTA, 5 μg sheared salmon sperm DNA, and 2 μg of linearized transforming DNA, in a final volume of 500 μL. This mixture was incubated at 30° C. for 1 hour, followed by 42° C. for 30 minutes. The cells were then pelleted and resuspended in liquid YPD media and permitted to grow for 3 hours at 30° C. or overnight at 22° C. to allow for expression of the HygR antibiotic resistance gene before plating on the selective media, containing 100 μg/ml hygromycin. Most of the DNA sequences used herein are codon-optimized for expression in the respective host system and as indicated in the sequence listing.

DNA molecular biology. Plasmid MB10306 (SEQ ID NO:5) and MB10569 (SEQ ID NO:6) containing expression systems for DrBCO, LmATF, and FfRDH was synthesized at Genscript (Piscataway, NJ, USA). Plasmid MB10306 contains both the ‘URA3’ and ‘HOM3” markers for selection in Yarrowia lipolytica transformations. For clean gene insertion by random nonhomologous end joining of the gene and marker SfiI plasmid fragment (of MB10306, or other plasmids in Table 1) of interest was purified by gel electrophoresis and Qiagen gel purification column. Clones were verified by sequencing. Typically, genes are synthesized at GenScript (Piscataway, NJ), with introduction of amino acid substitutions according to Table 1 (column “mutation”). Transformants were screened for homoserine auxotrophy and subsequently sequenced using primers flanking the HOM3 sequence and clean frameshifts were selected to move forward. Expression of mutant ATFs in Saccharomyces cerevisiae is described in Example 1 of WO2020141168.

Sequences. Plasmids comprising the respective LmATF and LmATF* according to SEQ ID NO:2 and 4 (polynucleotide according to SEQ ID NO:4 corresponds to nucleic acid encoding LmATF1 originated from L. mirantina shown in SEQ ID NO:2 in WO2020141168) as well as the modified enzymes including specific amino acid substitutions are listed in Table 1 and/or the sequence listing, with codon-optimized sequences for expression in Yarrowia lipolytica or Saccharomyces cerevisiae specifically indicated.

TABLE 1
list of plasmids used for construction of the strains carrying
the non-modified or modified heterologous Yarrowia lipolytica
codon-optimized ATF-genes from Lachancea mirantina as insert.
With the exception of MB10569, MB10597 and MB10599 (based
on LmATF*), all inserts are based on LmATF according to
SEQ ID NO: 1. For more details, see text.

| Plasmid | Insert |
|---|---|
| MB10306 | LmATF |
| MB10569 | LmATF* |
| MB10604 | LmATF_A451L |
| MB10605 | LmATF_A451M |
| MB10608 | LmATF_T473A |
| MB10609 | LmATF_L483M |
| MB10610 | LmATF_T473A_A451L |
| MB10611 | LmATF_T473A_A451M |
| MB10612 | LmATF_T473A_L483M |
| MB10613 | LmATF_A451L_L483M |
| MB10614 | LmATF_A451M_L483M |
| MB10615 | LmATF_T473L_L483M |
| MB10616 | LmATF_T473A_A451L_L483M |
| MB10617 | LmATF_T473L_A451L_L483M |
| MB10618 | LmATF_T473A_A451M_L483M |
| MB10619 | LmATF_T473L_A451M_L483M |
| MB10620 | LmATF_T473A_A451L_L483M_L452F |
| MB10621 | LmATF_T473L_A451L_L483M_L452F |
| MB10622 | LmATF_T473A_A451M_L483M_L452F |
| MB10623 | LmATF_T473L_A451M_L483M_L452F |
| MB10644 | LmATF_T473A_A451L_L483M_L452F_Q68L_N512F |
| MB10645 | LmATF_T473L_A451L_L483M_L452F_Q68L_N512F |
| MB10646 | LmATF_T473A_A451M_L483M_L452F_Q68L_N512F |
| MB10647 | LmATF_T473L_A451M_L483M_L452F_Q68L_N512F |
| MB10597 | LmATF*_T473A |
| MB10599 | LmATF*_L483M |

UPLC reverse phase retinol method. For rapid screening this method does not separate cis-isomers, only major functional groups. A Waters Acquity UPLC with PDA detection (or similar) with auto sampler was used to inject samples. An Acquity UPLC HSS T3 1.8 um P/N 186003539 was used to resolve retinoids. The mobile phase consisted of either, 1000 mL hexane, 30 mL isopropanol, and 0.1 mL acetic acid for retinoid related compounds. The flow rate for each was 0.6 mL per minute. Column temperature was 20° C. The injection volume was 5 NL. The detector was a photodiode array detector collecting from 210 to 600 nm. Analytes were detected according to Table 2.

TABLE 2A
list of analytes using reverse phase retinol method. The addition
of all added intermediates gives the total amount retinoids. Beta-
carotene* can be detected in 325 nm and will interfere with retinyl
ester quantitation, therefore care must be taken to observe the
carotene peak and not include them in the retinoid quantification.
“N/A” means “not available”. For more details, see text.

| | Retention | Lambda max | Response |
|---|---|---|---|
| Intermediates | time [min] | [nm] | factor |
| retinyl-acetate | 2.93 | 325 | 1.00 |
| retinyl-esters | 3.2-3.8 | 325 | 1.68 |
| retinal | 2.77 | 325 | 0.87 |
| retinol | 2.73 | 325 | 0.87 |
| Beta-carotene* | 3.56 | 450 | N/A |

TABLE 2B
UPLC Method Gradient with solvent A: water; solvent B: acetonitrile;
solvent C: methanol; solvent D: tert-butyl methyl ether.

| Time | | | | | Flow | Pressure |
|---|---|---|---|---|---|---|
| [min] | % A | % B | % C | % D | [ml/min] | [psi/bar] |
| 0 | 50 | 50 | 0 | 0 | 0.5 | 9500-14000max |
| 0.5 | 50 | 50 | 0 | 0 | 0.5 | |
| 1.0 | 0 | 50 | 50 | 0 | 0.5 | |
| 1.25 | 0 | 0 | 100 | 0 | 0.5 | |
| 3.25 | 0 | 0 | 5 | 95 | 0.5 | |
| 3.5 | 0 | 0 | 5 | 95 | 0.5 | |
| 4.0 | 0 | 0 | 100 | 0 | 0.5 | |
| 4.25 | 0 | 50 | 50 | 0 | 0.5 | |
| 4.5 | 50 | 50 | 0 | 0 | 0.5 | |

Method Calibration. Method is calibrated on retinyl acetate, retinols and retinals are quantitated against retinyl-acetate using the indicated response factor.

Retinyl Acetate is dissolved in THF at ~200 μg/ml for stock solution using a volumetric flask. Using volumetric flasks, ×20, ×50 and ×100 dilutions of stock solution in 50/50 methanol/MTBE were made. UV absorbance of retinyl acetate becomes nonlinear fairly quickly, so care must be taken to stay within the linear range. Consequently, lower concentrations might be better. Retinyl palmitate can also be used as retinyl ester calibration. Peaks for retinyl acetate at about 3 minutes and peaks for retinyl esters (long-chain retinyl esters) at around 3.5 minutes.

Sample preparation. Samples were prepared by various methods depending on the conditions. For whole broth or washed broth samples the broth was placed in a Precellys® tube, weighed, and mobile phase was added. Briefly in a 2 ml Precellys® tube, add 25 μl of well mixed broth and 975 μl of THF. The samples were then processed in a Precellys® homogenizer (Bertin Corp, Rockville, MD, USA) on the highest setting 3× according to the manufacturer's directions, typically 3×15×7500 tpms. For the washed pellet the samples were spun in a 1.7 ml tube in a microfuge at 10000 rpm for 1 minute, the broth decanted, 1 ml water added, mixed, pelleted and decanted, and brought up to the original volume. The mixture was pelleted again and brought up in appropriate amount of mobile phase and processed by Precellys® bead beating. For analysis of silicone oil fraction, the sample was spun at 4000 RPM for 10 minutes and the oil was decanted off the top by positive displacement pipet (Eppendorf, Hauppauge, NY, USA) and diluted into mobile phase mixed by vortexing and measured for retinoid concentration by UPLC analysis.

Fermentation conditions in Yarrowia. Fermentations were identical to the previously described conditions using preferably a silicone oil overlay and stirred tank that was preferably glucose in a bench top reactor with 0.5 L to 5 L total volume (see WO2016172282). Generally, the same results were observed with a fed batch stirred tank reactor with an increased productivity demonstrating the utility of the system for the production of retinoids. Preferably, fermentations were batched with 5% glucose and 20% silicone oil was added after dissolved oxygen dropped below about 20% and feed was resumed to achieve 20% dissolved oxygen throughout the feeding program.

### Example 2: Production of Retinyl Acetate in Yarrowia lipolytica Expressing Mutant LmATF

For expression of heterologous ATF in Yarrowia lipolytica as a host, the strain ML15710 (see Ex. 5 in WO2016172282) was transformed with plasmid MB9287 (see Ex. 1 in WO2022090548) to isolate a lip2 lip3 lip8 mutant derivative. This derivative was selected on 5-fluoorotic acid to isolate a uracil auxotroph, designated as strain ML18667-new. This strain was transformed with a plasmid listed in Table 1 above, each of which consists of the indicated ATF allele, DrBCO, and FfRDH12. Transformants of ML18667-new with SfiI-linearized plasmids from Table 1 were selected for uracil prototrophy. Transformants were grown in shake plates as described in Example 1, and the percentage of retinyl acetate (retinyl acetate/total retinoids) using the mutant ATFs in relation to the percentage of retinyl acetate using the reference LmATF expressed on plasmid MB10603 (set at 100%; SEQ ID NO:5) or LmATF* expressed on plasmid MB10569 is shown in Table 3.

TABLE 3A
acetylation of retinol into retinyl acetate (“retAc”)
as enhanced by action of modified ATFs (all based on LmATF
according to SEQ ID NO: 2). For more details, see text or Table 1.

| Plasmid | retAc [%] |
|---|---|
| MB10603 | 100 |
| MB10604 | 233 |
| MB10605 | 122 |
| MB10608 | 268 |
| MB10609 | 265 |
| MB10610 | 272 |
| MB10611 | 229 |
| MB10612 | 549 |
| MB10613 | 313 |
| MB10614 | 215 |
| MB10615 | 256 |
| MB10616 | 668 |
| MB10617 | 279 |
| MB10618 | 600 |
| MB10619 | 204 |
| MB10620 | 548 |
| MB10621 | 342 |
| MB10622 | 583 |
| MB10623 | 246 |
| MB10644 | 205 |
| MB10645 | 108 |
| MB10646 | 231 |
| MB10647 | 134 |

Each of the indicated mutations improved the percentage of retinyl acetate when compared to the reference sequence according to SEQ ID NO:1 by 8% up to over 560%.

TABLE 3B
acetylation of retinol into retinyl acetate (“retAc”)
as enhanced by action of modified ATFs (all based on LmATF*
according to SEQ ID NO: 4). For more details, see text or Table 1.

| Plasmid | retAc [%] |
|---|---|
| MB10596 | 100 |
| MB10597 | 156 |
| MB10599 | 177 |

Each of the indicated mutations improved the percentage of retinyl acetate when compared to the reference sequence according to SEQ ID NO:3 by 56 to 77%.

1-14. (canceled)

15. A modified acetyl transferase EC 2.3.1.84] with activity towards acetylation of retinol into retinyl acetate, wherein upon expression of said modified enzyme in a suitable retinol-producing Yarrowia strain the percentage of retinyl acetate is increased by at least 10-20 wt % as compared to the process using the same conditions but expressing the respective or corresponding enzyme according to SEQ ID NO:1, said modified acetyl transferase:
(a) is based on an enzyme with at least 45% sequence identity to Lachancea mirantina ATF1 according to SEQ ID NO:1,
(b) comprising a 7 amino acid motif N-H-x(3)-D-[GA], wherein “x” denotes an arbitrary amino acid, and wherein said motif corresponding to position N218 to G224 in the polypeptide according to SEQ ID NO:1;
(c) comprising at least one amino acid substitution in an amino acid residue corresponding to position 451, 473, and/or 483 in the polypeptide according to SEQ ID NO:1, said amino acid substitution being selected from T473A, L483M, A451L, and/or A451M;
with the proviso that amino acid residue corresponding to position 69 in SEQ ID NO:1 is either histidine or alanine, amino acid residue corresponding to position 407 in SEQ ID NO:1 is either valine or isoleucine, amino acid residue corresponding to position 409 in SEQ ID NO:1 is either glycine or alanine, amino acid corresponding to position 480 is either serine or glutamine, amino acid residue corresponding to position 484 in SEQ ID NO:1 is either isoleucine or leucine.

16. The modified acetyl transferase according to claim 15 with activity towards acetylation of retinol with a percentage of at least 81% based on total retinoids when expressed in a suitable retinol-producing Yarrowia strain and as compared to expression of the respective enzyme according to SEQ ID NO:1 or SEQ ID NO:3, said modified acetyl transferase.

17. The modified acetyl transferase according to claim 15, further comprising at least one amino acid substitution in an amino acid residue corresponding to position 68, 452 and/or 512 in the polypeptide according to SEQ ID NO:1, wherein said amino acid corresponding to position 68 being replaced by leucine, said amino acid corresponding to position 452 and/or position 512 in SEQ ID NO:1 being replaced by phenylalanine.

18. The modified enzyme according to claim 15, furthermore comprising one or more amino acid substitution(s) at position(s) corresponding to amino acid residues selected from H69, V407, G409, S480 and/or I484 in the polypeptide according to SEQ ID NO:1, wherein the histidine on position corresponding to 69 in SEQ ID NO:1 is replaced by alanine, asparagine or serine, and/or wherein the valine on position corresponding to 407 in SEQ ID NO:1 is replaced by isoleucine, and/or wherein the glycine on position corresponding to 409 in SEQ ID NO:1 is replaced by alanine, and/or wherein the serine on position corresponding to 480 in SEQ ID NO:1 is replaced by glutamic acid, phenylalanine, leucine, methionine or glutamine, and/or wherein the isoleucine on position corresponding to 484 in SEQ ID NO:1 is replaced by leucine.

19. The modified enzyme according to claim 15 comprising at least one of the following amino acid substitutions, wherein the positions correspond to amino acid residues in a polypeptide according to SEQ ID NO:1: T473A_A451L, T473A_A451M, T473A_L483M, A451L_L483M, A451M_L483M, T473L_L483M, T473A_A451L_L483M, T473L_A451L_L483M, T473A_A451M_L483M, T473L_A451M_L483M, T473A_A451L_L483M_L452F, T473L_A451L_L483M_L452F, T473A_A451M_L483M_L452F, T473L_A451M_L483M_L452F, LmATF_T473A_A451L_L483M_L452F_Q68L_N512F, T473A_A451M_L483M_L452F_Q68L_N512F, T473L_A451M_L483M_L452F_Q68L_N512F.

20. The modified enzyme according to claim 15, wherein the percentage of retinyl acetate based on total retinoids obtained from catalytic acetylation of retinol is increased by at least 10 to 20% compared to catalytic acetylation reaction using the corresponding non-modified enzyme according to SEQ ID NO:1 or SEQ ID NO:3.

21. The modified enzyme according to claim 15, which is expressed in a retinol-producing host cell expressing genes involved in catalysis of retinal into retinol and/or beta-carotene into retinal.

22. A retinoid-producing host cell expressing an enzyme according to claim 15.

23. The host cell according to claim 22 which is a fungal host cell, preferably selected from Yarrowia or Saccharomyces.

24. The host cell according to claim 21, further expressing enzymes involved in the mevalonate pathway and/or carotenoid pathway for generation of beta-carotene, retinal and retinol.

25. The host cell according to claim 24, wherein the enzyme catalyzing the conversion of beta-carotene into retinal is a beta carotene oxygenase selectively producing trans-retinal with a percentage of at least 95% trans-retinal based on total retinoids comprising cis- and trans-retinal.

26. A process for the production of retinoids comprising retinal, retinol and retinyl acetate, comprising cultivation of the host cell according to claim 21 under suitable culture conditions with the carbon source being selected from glucose, fructose, raffinose, lactose, galactose, glycerol, xylose, arabinose, sucrose, maltose, ethanol or mixtures thereof and in the presence of a lipophilic substance, wherein the percentage of retinyl acetate generated during said process is at least 81% based on total retinoids.

27. The process according to claim 27, wherein the lipophilic substance is selected from synthetic or natural oils or isoparaffins.

28. The process according to claim 26, wherein the percentage of retinyl acetate based on total retinoids is increased by at least 10 to 500% compared to a process using an ATF1 according to SEQ ID NO:1 or SEQ ID NO:3.


SEQUENCE LISTING (6 sequences)

SEQ ID NO: 1 - protein, 559 aa, synthetic construct
MGDLDARGTSAHPELSERPSIMPSMSDIQDPSGDDKATPRGSAAGLPQLELAGHARRLGHLENFFAVQARQQIYSSFAVFCEFDTACSLAQLASAVRNVCLSNPLLLHTVEPKHPDIAGFYHSDEYLSRPWPQHDYMRVLREVHVADVVMNGQKEHAHVVRDAVDVFQAHGNQVTSELLELMTQIEIPHASQTRPSWRLLCFPHGEANRWRTFAFVSNHCSSDGLSGLNFFRDLQKELAHGPTSGAPGAPGASGVIFDYAQDAATLPKLPPPIDQKLDYRPSKKALLGLLAGKFVREKLGYVSAAPPTTPTSDLAHPEGHQYYCYLVNVPTSSVAHIKTQVRENVPHKCTLTPFLQACWLVSLFKYGRVFSGSWLERYTDVLVAMNTRQLLPEDLELQRQYRYGSNIGAVRYNYPIAPLDVRDNDQKFWSLVESYRLALSDARDKNDYLYALGALMLPEIYEKKNVDAVVNDTILNQRRQGTLLSNVGYVRDEQPTAFAIKNHVFSQGVGANRNAFVLNICATDQGGLNIAISIAKGTLASRQEGQELCDIFKSTLLRF

SEQ ID NO: 2 - DNA, 1680 bp, synthetic construct
  source 1..1680: mol_type = other DNA
atgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccaggctcgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacattggagctgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgacagggaacccttctttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaa

SEQ ID NO: 3 - protein, 559 aa, Lachancea mirantina
MGDLDARGTSAHPELSERPSIMPSMSDIQDPSGDDKATPRGSAAGLPQLELAGHARRLGHLENFFAVQHRQQIYSSFAVFCEFDTACSLAQLASAVRNVCLSNPLLLHTVEPKHPDIAGFYHSDEYLSRPWPQHDYMRVLREVHVADVVMNGQKEHAHVVRDAVDVFQAHGNQVTSELLELMTQIEIPHASQTRPSWRLLCFPHGEANRWRTFAFVSNHCSSDGLSGLNFFRDLQKELAHGPTSGAPGAPGASGVIFDYAQDAATLPKLPPPIDQKLDYRPSKKALLGLLAGKFVREKLGYVSAAPPTTPTSDLAHPEGHQYYCYLVNVPTSSVAHIKTQVRENVPHKCTLTPFLQACWLVSLFKYGRVFSGSWLERYTDVLVAMNTRQLLPEDLELQRQYRYGSNVGGVRYNYPIAPLDVRDNDQKFWSLVESYRLALSDARDKNDYLYALGALMLPEIYEKKNVDAVVNDTILNQRRSGTLISNVGYVRDEQPTAFAIKNHVFSQGVGANRNAFVLNICATDQGGLNIAISIAKGTLASRQEGQELCDIFKSTLLRF

SEQ ID NO: 4 - DNA, 1680 bp, Lachancea mirantina
  source 1..1680: mol_type = genomic DNA
atgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccagcaccgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacgttggaggtgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgatctggaacccttatttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaa

SEQ ID NO: 5 - DNA, 14191 bp, synthetic construct
  source 1..14191: mol_type = other DNA
gcggagcctaggccggccagcgtggcgcgtggccatattggccagcagcttactacagctcagccaaagtggatcatgatggacggaaatatcggactggaggccaaaaaggaggtgctcaaatacgcacgggacaagtctgcacaggtggcattcgaacccacgtctgtccccaaagccgctgctctttccgagctcaatctgcccgtttaccccaacaactccattgccttagccacccccaacactgcagagctcgaggccatgttcgaggcattccacgagaagggccgatttgacgttgacgactggtttccagtcatagacggtcttgctctcggagctgactttagaaacggagccaccatgttgtcgcaccaacatcgtggactcaaagctattctcgaacaaggaactctggcccaggcaatccacatgctaccatacatcccaactttgatcatcaagggcggagccaacggcgtcgtcgtcttccaactcattgatgatatcgagtctgccattcattcacagcgttctgcttccaataaaacgcctggcctgttccagaagggcaacgctgcgagtggaaacaccaaggtcggcgtctacatgcagtactttgagcccgaagaagtgggcagtcagtcgattgtaagcgtgactggagctggagacactctgtttggaaccctggccatggagattgtcaaggacgagtcctggttgaacgatatgggagacaagaagagtgcagttgtttctcgagccatgaacaatgctgtgaagactattcagagtaaggacgctgtctgcaagagcatcctttaagtgatttgccatgctttctcttcttccacgatgtaaatacttattttacacactactgtgcagtagcaaatacagaacaagagttgtcgcctattgacagtacagtacgagtagtgtatgtacagtagttatacaatatctatgtgaaattcgtcggcagctttcggctgatgaactacgagttcttcgtaataaatcatcaacgtaataagcttggtaccttgacaaatagctcaagggcgtggagcacagggtttaggaggttttaatgggcgagaaggcgcgtagatgtagtcttcctcggtcccatcggtaatcacgtgtgtgccgatttgcaagacgaaaagccacgagaataaaccgggagaggggatggaagtccccgaacagcaaccagcccttgccctcgtggacataacctttcacttgccagaactctaagcgtcaccacggtatacaagcgcacgtagaagattgtggaagtcgtgttggagactgttgatttgggcggtggaggggggtatttgagagcaagtttgagatttgtgccattgagggggaggttattgtggccatgcagtcggatttgccgtcacgggaccgcaacatgcttttcattgcagtccttcaactatccatctcacctcccccaatggcttttaactttcgaatgacgaaagcacccccctttgtacagatgactatttgggaccaatccaatagcgcaattgggtttgcatcatgtataaaaggagcaatcccccactagttataaagtcacaagtatctcagtatacccgtctaaccacacatttatcacaatgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccaggctcgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacattggagctgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgacagggaacccttctttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatctgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcgtagaccccggtgtatataaaggggtggagaggacggattattagcaccaacacacacacttatactacatgctagccacaaaaatgctctctttcttctggcgaaacggtatcgagactcccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagactcctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaagttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaaaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcacactattatcacattactacatccaaacccctacagggggaggagctctccaatcaaatatgtacattaactatctctcgtaaatcattgttataagaccgtccgtgactgtctaaatcggttcattcgttgtaacaaatcagtgtaacaactcgtggtacgcccggtttgcttcgctcacgctcccctagattttccgtctaggacacaacaagccctggtggatgacgttcaacgccttcagcgcgtctttttcctcaataacacacgagatgttaatttcgttggctccctgagaaatcatctcaatgttaatacctgcctgggccagagtagagaagaacatgcctgcacatccgaccatggccttcattctcgttccgatcagcgacacaatggtcatgcctcgcttcacatctactgtgccgtactttcgcagctcctccacagcctgcttgaggttggagtcgggagcatggaaggccatcgacacatggacctcagaagtggagatgagatcgacgacaagtttctgctggtcgagagtagcaaagatcttgttcaagaagccatgactcttggttcgcttattcgaatgaacgttgagaacggtgatattcgatttggtagtgacagctgtaggcttcttctcgtcatctacaggagcagacacctggggagtgaatgagccagaggacagagaagttagcgagtcggtggaagaagccagatcctggacattgtttttcgttttcaggttgcccgagccgtcagcagatgggtagatgatagttcctcctcccagggggttttccacgtttttgattcggataggaatatgggccttaatgacctgctccatggtgaaggggtggatgacttcagatccgtagtaggtcaactcagcggcctcctcgggggtaatgatgggcagcagacgggcagtggacacctttctgggatcggcagtgaagacaccatcgacttctttccagatctgaagctccttggcgtccagacccacagccaccagagcagcacacagatcggtgtatcctcgtccgatctggcttaggaggcctcccttgacggggccaaagaaaccggtcaacacgggcaccatgtttcccttgacaggagcagagtccgtgttaggagagccgggaagagtgatgatctcgcccagaacacgtccgaggtccgaatagaacttgggatcttcggcattggtggtggtaactgcgtgagacaggtcaaagtaccgtgcgttaacaccagcgtcccgcataactgcagtcatgtacatgcaactgagcttctccccaatggccatgatggagtcgagtgttcggggggagatctcagaaatgatttcagcagcagccaggattcgcaacagctggtcgcactcgccgttgatgtcggcattgaggttttcgagcagttcggggttcttgacgtcacgcttagcagctgcaagatggtcttctcggatagactcaatgatggggttgtaggcgtctgatccgagaagagcagagtcagcagcagcaataagacgggtggtggttccctcggccttggtggcctctagatggcctccttggccccattccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

SEQ ID NO: 6 - DNA, 14006 bp, synthetic construct
  source 1..14006: mol_type = other DNA
gcggagcctaggccggccagcgtggcgcgtggccatattggccagcagcttactacagctcagccaaagtggatcatgatggacggaaatatcggactggaggccaaaaaggaggtgctcaaatacgcacgggacaagtctgcacaggtggcattcgaacccacgtctgtccccaaagccgctgctctttccgagctcaatctgcccgtttaccccaacaactccattgccttagccacccccaacactgcagagctcgaggccatgttcgaggcattccacgagaagggccgatttgacgttgacgactggtttccagtcatagacggtcttgctctcggagctgactttagaaacggagccaccatgttgtcgcaccaacatcgtggactcaaagctattctcgaacaaggaactctggcccaggcaatccacatgctaccatacatcccaactttgatcatcaagggcggagccaacggcgtcgtcgtcttccaactcattgatgatatcgagtctgccattcattcacagcgttctgcttccaataaaacgcctggcctgttccagaagggcaacgctgcgagtggaaacaccaaggtcggcgtctacatgcagtactttgagcccgaagaagtgggcagtcagtcgattgtaagcgtgactggagctggagacactctgtttggaaccctggccatggagattgtcaaggacgagtcctggttgaacgatatgggagacaagaagagtgcagttgtttctcgagccatgaacaatgctgtgaagactattcagagtaaggacgctgtctgcaagagcatcctttaagtgatttgccatgctttctcttcttccacgatgtaaatacttattttacacactactgtgcagtagcaaatacagaacaagagttgtcgcctattgacagtacagtacgagtagtgtatgtacagtagttatacaatatctatgtgaaattcgtcggcagctttcggctgatgaactacgagttcttcgtaataaatcatcaacgtaataagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccagcaccgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacgttggaggtgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgatctggaacccttatttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatctgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcgtagaccccggtgtatataaaggggtggagaggacggattattagcaccaacacacacacttatactacatgctagccacaaaaatgctctctttcttctggcgaaacggtatcgagactcccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagactcctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaagttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaaaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcacactattatcacattactacatccaaacccctacagggggaggagctctccaatcaaatatgtacattaactatctctcgtaaatcattgttataagaccgtccgtgactgtctaaatcggttcattcgttgtaacaaatcagtgtaacaactcgtggtacgcccggtttgcttcgctcacgctcccctagattttccgtctaggacacaacaagccctggtggatgacgttcaacgccttcagcgcgtctttttcctcaataacacacgagatgttaatttcgttggctccctgagaaatcatctcaatgttaatacctgcctgggccagagtagagaagaacatgcctgcacatccgaccatggccttcattctcgttccgatcagcgacacaatggtcatgcctcgcttcacatctactgtgccgtactttcgcagctcctccacagcctgcttgaggttggagtcgggagcatggaaggccatcgacacatggacctcagaagtggagatgagatcgacgacaagtttctgctggtcgagagtagcaaagatcttgttcaagaagccatgactcttggttcgcttattcgaatgaacgttgagaacggtgatattcgatttggtagtgacagctgtaggcttcttctcgtcatctacaggagcagacacctggggagtgaatgagccagaggacagagaagttagcgagtcggtggaagaagccagatcctggacattgtttttcgttttcaggttgcccgagccgtcagcagatgggtagatgatagttcctcctcccagggggttttccacgtttttgattcggataggaatatgggccttaatgacctgctccatggtgaaggggtggatgacttcagatccgtagtaggtcaactcagcggcctcctcgggggtaatgatgggcagcagacgggcagtggacacctttctgggatcggcagtgaagacaccatcgacttctttccagatctgaagctccttggcgtccagacccacagccaccagagcagcacacagatcggtgtatcctcgtccgatctggcttaggaggcctcccttgacggggccaaagaaaccggtcaacacgggcaccatgtttcccttgacaggagcagagtccgtgttaggagagccgggaagagtgatgatctcgcccagaacacgtccgaggtccgaatagaacttgggatcttcggcattggtggtggtaactgcgtgagacaggtcaaagtaccgtgcgttaacaccagcgtcccgcataactgcagtcatgtacatgcaactgagcttctccccaatggccatgatggagtcgagtgttcggggggagatctcagaaatgatttcagcagcagccaggattcgcaacagctggtcgcactcgccgttgatgtcggcattgaggttttcgagcagttcggggttcttgacgtcacgcttagcagctgcaagatggtcttctcggatagactcaatgatggggttgtaggcgtctgatccgagaagagcagagtcagcagcagcaataagacgggtggtggttccctcggccttggtggcctctagatggcctccttggccccattccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag